(19) United States
(12) Patent Application Publication
(10) Pub. No.: US 2014/0235996 A1
(43) Pub. Date: Aug. 21, 2014

(54) METHOD FOR NON-INVASIVE MAPPING OF MYOCARDIAL ELECTRIC ACTIVITY

(71) Applicant: Korea Research Institute of Standards and Science, Daejeon (KR)

(72) Inventors: Kiwoong Kim, Daejeon (KR); Hyukchan Kwon, Daejeon (KR); Yong-Ho Lee, Daejeon (KR); Kwon-Kyu Yu, Daejeon (KR); Jin-Mok Kim, Daejeon (KR)

(73) Assignee: KOREA RESEARCH INSTITUTE OF STANDARDS AND SCIENCE, Daejeon (KR)

(21) Appl. No.: 14/260,750

(22) Filed: Apr. 24, 2014

Related U.S. Application Data
(63) Continuation of application No. PCT/KR2012/008425, filed on Oct. 16, 2012.

(30) Foreign Application Priority Data
Oct. 26, 2011 (KR) 10-2011-0109926

(51) Int. Cl. A61B 5/04; A61B 6/03; A61B 5/055

(52) U.S. Cl. CPC A61B 5/04011; A61B 5/055; A61B 6/03
USPC 600/411; 600/512

(57) ABSTRACT

A method for mapping of myocardial electric activity includes measuring electrocardiogram data or magnetocardiogram data and mapping the degree of electric activity of a myocardial surface using the electrocardiogram data or the magnetocardiogram data. A signal source of the electrocardiogram data or the magnetocardiogram data is a myocardial surface potential that is scalar quantity. The mapping uses a lead-field vector which represents the sensitivity between the myocardial surface potential and the electrocardiogram or magnetocardiogram data, and a modified lead-field vector which combines a constraint matrix with a constraint condition where no potential sources exist in a specific region.

### CROSS-REFERENCE TO RELATED APPLICATIONS

This application is a continuation of and claims priority to PCT/KR2012/008425 filed on Oct. 16, 2012, which claims priority to Korea Patent Application No. 10-2011-0109926, filed on Oct. 26, 2011, the entirety of which is incorporated by reference herein.

### BACKGROUND

1. Field of the Invention

The present invention described herein generally relates to methods for mapping of myocardial electric activity and, more particularly, to a method for estimating a position of myocardial electric activity with periodicity, such as reentry wave or ectopic excitation, from multi-channel data measured by electrocardiogram or magnetocardiogram.

2. Description of the Related Art

Many heart diseases are caused by reentry excitation or ectopic excitation of myocardium. Such a conduction abnormality develops atrial arrhythmia, tarchycardia, and heart failure that cause a stroke. Moreover, myocardial conduction abnormality is the mechanism of ventricular fibrillation that causes sudden cardiac death resulting from cardiac arrest.

Conventionally, in order to detect myocardial conduction abnormality, a catheter electrode is inserted through the aorta and vena cava of the thigh to measure endocardial potentials one by one while changing positions. Alternatively, a multi-channel electrode patch is attached to the epicardium during thoracotomy surgery to measure the endocardial potentials.

A non-invasive method includes electrocardiogram (ECG) multiple electrodes are attached to the thorax and limbs to measure potentials and magnetocardiogram (MCG) that myocardial electric activity is measured by an ultra-sensitive magnetic sensor such as a superconducting quantum interference device (SQUID) or an atomic magnetometer.

In the case of electrocardiogram and magnetocardiogram, the extraction of conduction abnormalities mathematically corresponds to an inverse problem solution based on measured data of multi-channel myocardial electric activity. That is, a current source is estimated to be localized.

The present invention relates to a method for extracting a current source based on the multi-channel measurement data of myocardial electric activity. There are many methods for extracting a current source through an inverse problem solution. A current source is basically assumed to be current dipoles at a single position or multiple of positions. The moment and direction of the current dipoles are estimated to optimally explain the distribution of measured potentials or magnetic fields.

If current dipoles are assumed to be a current source, a solution of minimum norm estimation method is obtained by solving a linear equation to the moment and direction of current dipoles where their positions are fixed. Nonlinear optimization (e.g., simplex, conjugate gradient, etc.) and global nonlinear optimization (e.g., genetic algorithm, simulated annealing, etc.) by randomized estimation are used to obtain the moment, direction, and position of current dipoles through an iterative trial.

A so-called electrophysiology (EP) test inspects the myocardial electric activity using a catheter. The EP test examines the myocardial electric activity by inserting the catheter into a human body and contacting an electrode on the endocardium. Since the EP test is invasive, it always involves the danger of surgery. In particular, a measurable region is limited to the endocardium. Although the catheter may be introduced into the left atrium and the left ventricle through the aorta, it cannot reach the right atrium and the right ventricle without perforating septum. Likewise, although the catheter may be introduced into the right atrium and the right ventricle through the vena cava, it cannot reach the left atrium and the left ventricle without perforating septum.

Furthermore, a patient or doctors may be exposed to an excessive radiation such as X-ray during surgery may take several hours to locate the electrode at a correct position. Especially, an additional magnetic position tracking system (e.g., Carto system) is needed for spatial mapping of myocardial electric activity, since a two-dimensional radiation image cannot provide spatial information on catheter position.

In the case of an epicardial electrode array, not only a patient has a great burden on thoracotomy surgery, but also an expert technique is required for electrode attaching. Moreover, the epicardial electrode array cannot be used in prognosis observation after surgery.

In the case of non-invasive current mapping, the position of a current source is estimated by solving the inverse problem using multi-channel electrocardiogram or multi-channel magnetocardiogram. However, the non-invasive current mapping is estimating a current source by solving an ill-posed inverse problem based on non-invasive measurement data. Therefore, the estimation of a weak current source or deep current source reveals significant errors. As a result, the non-invasive current mapping has limitations in clinical application.

### SUMMARY

Embodiments of the present invention provide a method for estimating a position of myocardial electric activity with periodicity, such as reentry wave or ectopic excitation, from multi-channel data measured by electrocardiogram or magnetocardiogram.

A method for mapping of myocardial electric activity according to an embodiment of the present invention may include measuring electrocardiogram data or magnetocardiogram data, and mapping the electric activity on the myocardial surface using the electrocardiogram data or the magnetocardiogram data. A signal source of the electrocardiogram data or the magnetocardiogram data is a myocardial surface potential that is scalar quantity. The mapping method uses a lead-field vector which represents the sensitivity between the myocardial surface potential and the EEG or MCG data, and a modified lead-field vector which combines a constraint matrix with a constraint condition where no potential sources exist in a specific region.

In an embodiment of the present invention, mapping the degree of electric activity may use a minimum variance spatial filter, and the constraint condition may suppress the influence of correlated sources located at other regions to prevent interference generated from the correlated sources toward target sources to be estimated by the minimum variance spatial filter.

In an embodiment of the present invention, mapping the degree of electric activity may include generating a surface mesh to use a boundary element method as an electric conductor model of a peripheral organ including the myocardium and thorax; calculating a lead field vector between the myocardial surface potential and the electrocardiogram or magnetocardiogram data; extracting a covariance matrix using multi-channel measured electrocardiogram or magnetocardiogram data; obtaining a constraint matrix by applying a constraint condition in which there is no potential source in a specific region; obtaining a modified lead field vector including the constraint condition from the lead field vector; and calculating electric activity power of a vertex of a myocardial surface using the modified lead field vector and the covariance matrix.

In an embodiment of the present invention, the method may further include at least one of measuring MRI or CT to include heart and thorax; forming an electric conductor model of patient's individualized heart and organ using MRI or CT data; and separating a localization-desired magnetocardiogram or electrocardiogram waveform using second or higher-order statistics such as independent component analysis.

In an embodiment of the present invention, mapping the degree of electric activity may further include at least one of forming a minimum variance spatial filter using the modified lead field vector; extracting the degree of electric activity of a surface potential using a minimum variance spatial filter in which the constraint condition is included; extracting a current source using the surface potential; calculating imaginary coherence between electric activities of multiple the surface potentials; and extracting an extracted position of a route of an abnormal circuit formed by the current source.

In an embodiment of the present invention, calculating a lead field vector may include calculating an organ surface potential generated by a unit potential of a myocardial mesh surface by a boundary element method; and calculating a potential at an electrocardiogram electrode or a magnetic field at a magnetocardiogram sensor from the organ surface potential by a boundary element method.

In an embodiment of the present invention, mapping the degree of electric activity may include forming a surface mesh to use a boundary element method in an electric conductor model of a peripheral organ including myocardium and thorax; calculating a lead field vector between the myocardial surface potential and the electrocardiogram or magnetocardiogram data; extracting a covariance matrix using the multi-channel measured electrocardiogram or magnetocardiogram data; obtaining a constraint matrix by applying a constraint condition in which there is no potential source in a specific region; obtaining a modified lead field vector including the constraint condition from the lead field vector; forming a minimum variance spatial filter using the modified lead field vector; and extracting the degree of electric activity of a surface potential using the minimum variance spatial filter in which the constraint condition is included.

In an embodiment of the present invention, mapping the degree of electric activity may include at least one of extracting a current source using the surface potential; calculating imaginary coherence between electric activities of multiple surface potentials; and extracting an ablation position of a route of an abnormal circuit formed by the current source.

### BRIEF DESCRIPTION OF THE DRAWINGS

The present invention will become more apparent in view of the attached drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the present invention.

FIG. 1 is a flowchart illustrating a method for mapping of myocardial electric activity according to an embodiment of the present invention.

FIGS. 2A and 2B are flowcharts illustrating a method for mapping of myocardial electric activity according to another embodiment of the present invention.

FIG. 3 illustrates a magnetocardiogram measuring device according to an embodiment of the present invention.

FIG. 4 illustrates counterclockwise periodic rotational excitation of myocardium according to an embodiment of the present invention.

FIG. 5 illustrates a myocardial surface potential calculated through a method for mapping myocardial electric activity according to the present invention using a magnetic field or magnetocardiogram data calculated at respective positions where magnetocardiogram sensors are disposed. The magnetic field is generated from variation of a myocardial potential of FIG. 4.

### DETAILED DESCRIPTION

A method for mapping of myocardial electric activity according to an embodiment of the present invention calculates the position of the myocardial electric activity using data measured by multi-channel sensors. This method may be applied to both multi-channel electrocardiogram and multi-channel magnetocardiogram measurement data. For the convenience, only the magnetocardiogram measurement data will be described.

A beamforming method, which is a minimum variance spatial filter method, does not estimates a current source at a specific time but estimates a current source from a covariance matrix obtained from multi-channel data for a certain period of time. Thus, the beamforming method is suitable to localize a current source with periodicity such as reentry wave. However, the beamforming method contains intrinsic drawbacks. The first drawback is that the direction of current dipoles should be known before calculating source power in a beamformer based on an equivalent current dipole model. If the direction of the current dipoles is estimated wrong, an error is very large in power calculation by the beamformer.

In case of electroencephalography (EEG) or magnetoencephalography (MEG) study in which a beamformer has been widely used, a current source generating a magnetic field is a pyramidal cell whose direction is well defined at a cerebrum cortex layer. Thus, the current source may be approximated as a current dipole.

However, in case of the heart, electric excitation propagates along myocardial fibers while successively forming a wavefront. Thus, the current dipole model is structurally unsuitable for the heart.

The second problem of the beamformer is that if multiple correlated signal sources exist, they cannot be spatially separated. In particular, if the signal sources are successively activated, they have strong correlation and almost cannot be separated from each other.

Accordingly, the present invention proposes a method for effectively localizing current sources or potential sources.

Exemplary embodiments of the present invention will now be described more fully with reference to the accompanying drawings, in which exemplary embodiments of the present invention are shown. Exemplary embodiments of the present invention may, however, be embodied in many different forms and should not be construed as limited to the embodiments set forth herein. Rather, these exemplary embodiments of the present invention are provided so that this description will be thorough and complete, and will fully convey the concept of exemplary embodiments of the present invention to those of ordinary skill in the art. In the drawings, the sizes and relative sizes of elements may be exaggerated for clarity. Like numerals refer to like elements throughout.

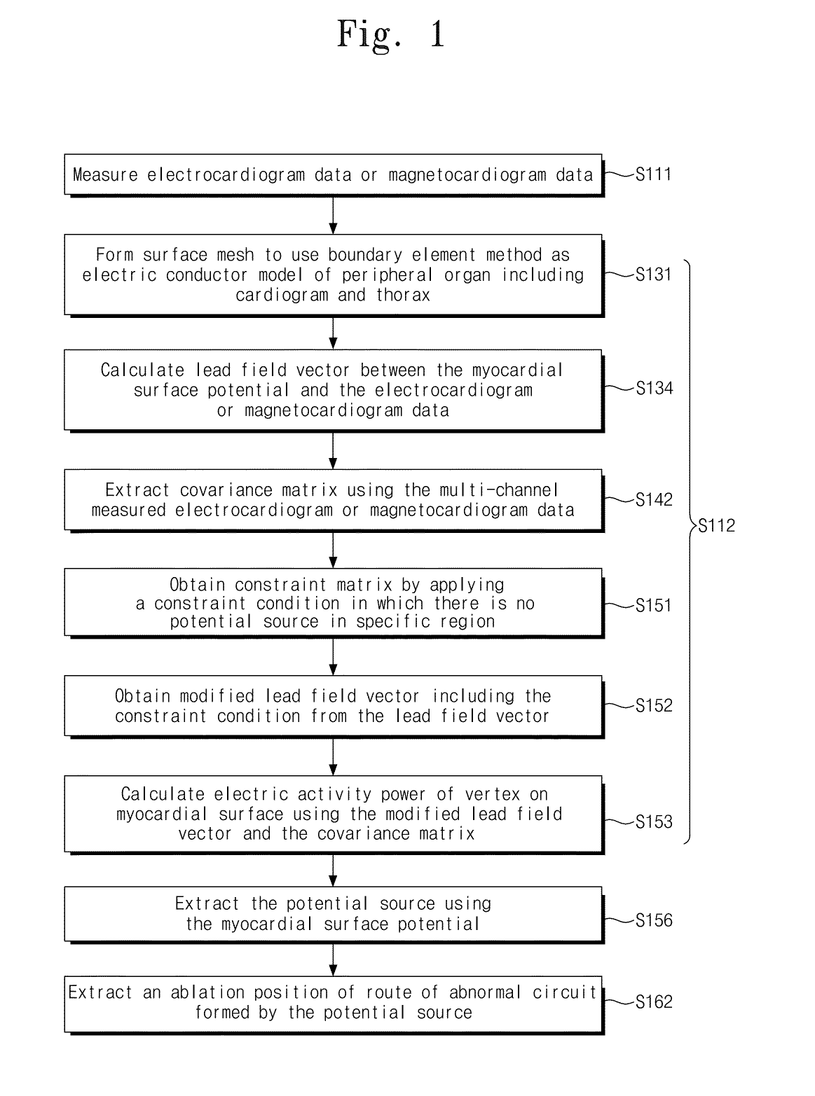

FIG. 1 is a flowchart illustrating a method for mapping of myocardial electric activity according to an embodiment of the present invention.

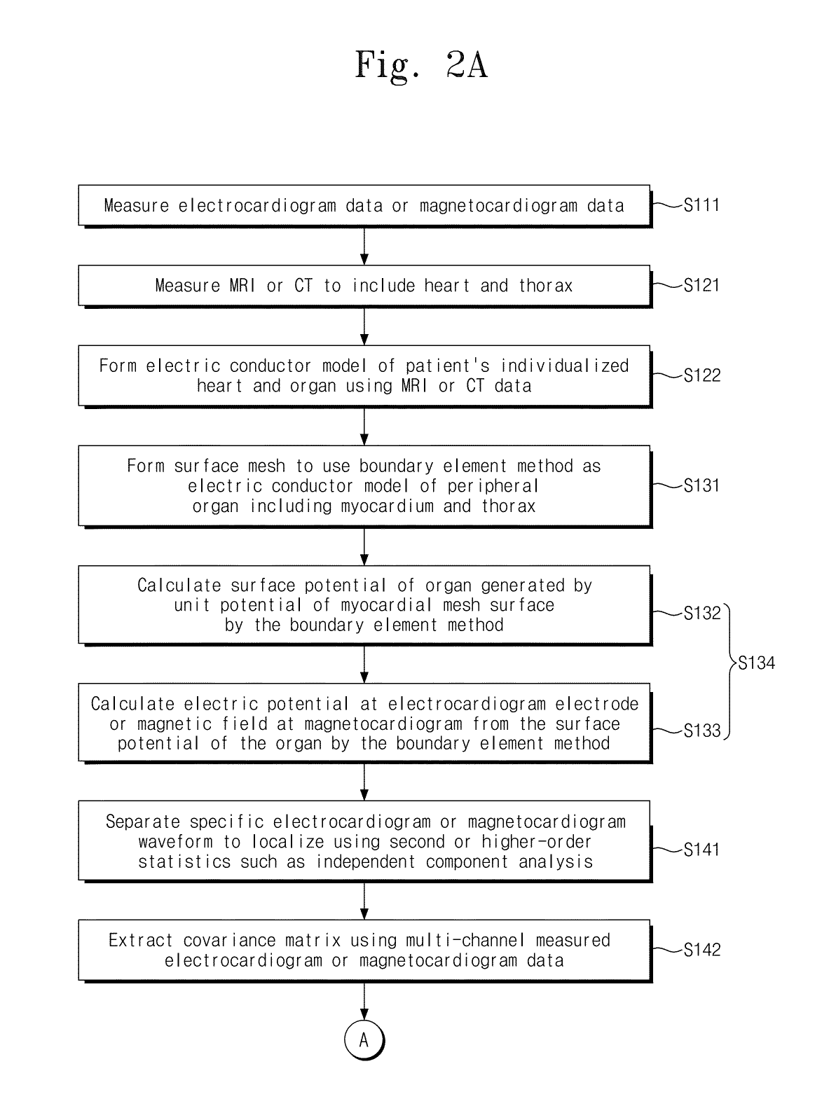

FIGS. 2A and 2B are flowcharts illustrating a method for mapping of myocardial electric activity according to another embodiment of the present invention.

Referring to FIG. 1 and FIGS. 2A and 2B, a method for mapping of myocardial electric activity includes measuring electrocardiogram data or magnetocardiogram data (S111) and mapping the degree of electric activity of a myocardial surface using the electrocardiogram data or the magnetocardiogram data (S112). A signal source of the electrocardiogram data or the magnetocardiogram data is a myocardial surface potential that is scalar quantity. The mapping uses a lead-field vector which represents the sensitivity between the myocardial surface potential and the electrocardiogram or magnetocardiogram data, and a modified lead-field vector which combines a constraint matrix with a constraint condition where no potential sources exist in a specific region.

The mapping step (S112) uses a minimum variance spatial filter. The constraint condition may suppress the influence of correlated sources located at other regions to prevent interference generated from the correlated sources toward target sources to be estimated by the minimum variance spatial filter.

The mapping step (S112) may include forming a surface mesh to use a boundary element method as an electric conductor model of a peripheral organ including the myocardium and thorax (S131), calculating a lead field vector between the myocardial surface potential and the electrocardiogram or magnetocardiogram data (S134), extracting a covariance matrix using the multi-channel measured electrocardiogram or magnetocardiogram data (S142), obtaining a constraint matrix by applying a constraint condition in which there is no potential source in a specific region (S151), obtaining a modified lead field vector including the constraint condition from the lead field vector (S152), and calculating electric activity power of a vertex of a myocardial surface using the modified lead field vector and the covariance matrix (S153).

According to a modified embodiment of the present invention, the mapping step (S112) may include forming a surface mesh to use a boundary element method as an electric conductor model of a peripheral organ including the myocardium and thorax (S131), calculating a lead field vector between the myocardial surface potential and the electrocardiogram or magnetocardiogram data (S134), extracting a covariance matrix using the multi-channel measured electrocardiogram or magnetocardiogram data (S142), obtaining a constraint matrix by applying a constraint condition in which there is no potential source in a specific region (S151), obtaining a modified lead field vector including the constraint condition from the lead field vector (S152), forming a minimum variance spatial filter using the modified lead field vector (S154), and extracting the degree of electric activity of a surface potential using the minimum variance spatial filter in which the constraint condition is included (S155).

The method for mapping of myocardial electric activity according to an embodiment of the present invention uses a surface potential source model (myocardial surface model) based on an equivalent double layer model instead of a current dipole model that is unsuitable as a myocardial current source. According to the equivalent double layer model (or surface potential source model), all current sources in a heart space may be equivalently expressed as a surface potential on the myocardium that surrounds the heart.

The myocardial surface potential source may generate a potential, which is affected by electric conductivity of peripheral organs, on the surface of a thoracic cage and the potential may be measured by electrocardiogram. Alternatively, the myocardial surface potential source may generate bioelectric current affected by electric conductivity of peripheral organs. The myocardial surface potential source may induce current, and a magnetic field generated by the current may be measured by a magnetocardiogram device.

Since a surface potential of the myocardium is a source, the method for mapping of myocardial electric activity has only to calculate single scalar quantity. Moreover, in a conventional minimum variance spatial filer, the entire heart space must be divided into three-dimensional mesh to estimate source power for vertices of all the meshes. However, the method for mapping myocardial electric activity may significantly reduce the amount of calculation because a source power estimation point is limited to vertices of a surface potential of a myocardial surface model.

When a value of potential source is known, a procedure of calculating an electrocardiogram measured value or a magnetocardiogram measured value is called a forward problem. Meanwhile, a procedure of obtaining a value of potential source from a measured value is called an inverse problem.

According to an embodiment of the present invention, an electric conductor model and a boundary element method (BEM) are used to calculate conduction current effect of peripheral organs or internal organs in the forward problem. More specifically, in the BEM, the electric conductor model used a surface mesh model including the heart consisting of two atria and two ventricles, a torso, and the lung.

A CT or MRI result is used to combine anatomical information of respective patients (S121). From the CT or MRI result, surfaces of patient's heart, torso, and lung are segmented to be triangularly meshed. The meshed surfaces of the respective organs form a closed surface (S122).

Values of the myocardial surface potential source are given to vertices of the triangle mesh constituting the heart, respectively. When a potential value varies by one unit at each vertex of a myocardial surface mesh constituting a surface of the heart, a vector-matrix version of the variation degree of a measured value at each measurement sensor channel of electrocardiogram or magnetocardiogram is called a lead field vector.

If a surface of a mesh of each organ is S^{k}, a potential φ(r) at a point r on a specific surface S^{l} is given by a boundary element method as Equation (1) below.

$$φ ( {r}^{->} ) = \frac{2 {σ}_{s}}{{σ}_{-}^{l} + {σ}_{+}^{l}} {φ}^{∞} - \frac{1}{2 π} {\sum }_{k = 1}^{K} \frac{{σ}_{-}^{k} - {σ}_{+}^{k}}{{σ}_{-}^{l} + {σ}_{+}^{l}} {\int }_{{S}^{k}} φ ( {{r}^{->}}^{′} ) \frac{( {r}^{->} - {{r}^{->}}^{′} )}{{\vert {r}^{->} - {{r}^{->}}^{′} \vert}^{3}} \cdot d {{S}^{->}}^{′} , {r}^{->} ∈ {S}^{l} \qquad Equation ( 1 )$$

In the Equation (1), σ₊^{k} and σ₋^{k} represent electric conductivities at the outside and the inside of a k^{th} surface, respectively; l represents an interested specific surface; φ^{∞} represents an initial potential; σₛ represents electric conductivity at a source position; K represents the number of closed surfaces constituting an organ; φ(r′) represents a potential at the point r′ on a surface S^{k}; σ₋^{l} represents electric conductivity at the inside of an l^{th} surface; and σ₊^{l} represents electric conductivity at the outside of the l^{th} surface.

A lead field vector between a surface potential on the myocardium and electrocardiogram or magnetocardiogram data may be calculated (S134).

The potential φ(r) of an organ surface, which is generated by a unit potential of a myocardial mesh surface, may be calculated using the first term in the right of the Equation (1).

In order to obtain the lead field vector, it is calculated while putting a unit potential in a vertex of the myocardial surface.

Referring to the Equation (1), potentials of surface meshes of respective organs may be calculated and a potential at a thoracic cage surface mesh is a measurement unit of electrocardiogram (S132).

A magnetic field may be calculated from the above-obtained potentials φ(r′) at the surface meshes of respective organs (S133).

$${B}^{->} = - \frac{{μ}_{0}}{4 π} {\sum }_{k = 1}^{K} ( {σ}_{-}^{k} - {σ}_{+}^{k} ) {\int }_{{S}^{k}} φ ( {{r}^{->}}^{′} ) d {{S}^{->}}^{′} \times \frac{( {r}^{->} - {{r}^{->}}^{′} )}{{\vert {r}^{->} - {{r}^{->}}^{′} \vert}^{3}} \qquad Equation ( 2 )$$

As a result, if a magnetocardiogram measurement magnetic field (or electrocardiogram measurement potential) is B, a potential value of a myocardial surface mesh is s, and a lead field vector L, then B=Ls.

The goal is to obtain the potential value s of the myocardial surface mesh from the measurement magnetic field B, i.e., is to be in the relationship of s=wB. A minimum variance spatial filter w is obtained by projecting the measurement magnetic field B to a spatial filter element that makes the best explanation for the potential value s of the myocardial surface. When the minimum variance spatial filter w is obtained, a modified lead field vector and a covariance matrix of actual measured values may be used. The measured values may be actually measured electrocardiogram or magnetocardiogram data.

However, activation patterns of multiple of potential sources may have correlation. In this case, the estimated locations of each source may be incorrectly calculated since the correlated potential sources interference with each other. In order to overcome this problem, a constraint condition in which there is no potential source in a specific region (e.g., there is no potential source in the ventricle when an atrial fibrillation potential source is estimated) may be applied. Thus, a precise activity position of potential source may be found out.

If a specific region in which there is no potential source includes Nc surface vertices, a constraint matrix (Lc) may be defined below (S151).

L_{c}=[L(r₍ₗ₎), . . . , L(r_{(N}_{_{c}}₎)] Equation (3)

In the Equation (3), L(r) represents a lead field vector at the vertex r where there is no source.

At this point, electric activity power P_{c}(r) of the vertex r on the myocardial surface may be calculated by Equation (4) below (S153).

\hat{P}_{c}(r)≡tr{[\tilde{L}^{T}(r)(C+εI)⁻¹\tilde{L}(r)]⁻¹} Equation (4)

In the Equation (4), C represents a covariance matrix of measured values; ε represents a normalizing constant that controls oversensitivity to noise; I represents a unit matrix; L(r) represents a modified lead field vector at the position r; tr represents a trace; and T represents a transpose.

The modified lead field vector L(r) is given by Equation (5) below (S152).

$$\tilde{L} ( r ) = [ \frac{l ( r )}{‖ l ( r ) ‖} , {L}_{c} ] \qquad Equation ( 5 )$$

In the Equation (5), ∥°∥ represents a Euclidean norm. Depth normalization may be done by dividing the lead field vector l(r) by its size at an obtaining-desired position r where there is a source. Since a potential source present in the deep is sensitive to a noise, an estimation error increases. The depth normalization may decrease this estimation error.

If the measurement magnetic field B makes an average of time series at respective measurement channels zero and a matrix is an m×N matrix (m being the number of measurement channels, and N being the number of time series samples), a covariance matrix C meets a relational expression as follows: C=BB^{T}/(N−1).

Conventionally, second-order statistical variables such as variance size are used to separate current sources. The second-order statistical variables may not be enough to separate P wave, QRS wave, and T wave that are specific waveforms of magnetocardiogram (MCG). Accordingly, independent component analysis employing higher-order statistical variables may be used beforehand to time-serially separate measured waveforms. After a localization-desired waveform is separated beforehand using second or higher-order statistics such as independent component analysis, a covariance matrix C may be calculated.

A minimum variance spatial filter w for obtaining time series of potential source variation is given by the Equation (6) below (S154).

w(r)≡[\tilde{L}^{T}(r)(C+εI)⁻¹\tilde{L}(r)]⁻¹\tilde{L}^{T}(r)(C+εI)⁻¹ Equation (6)

Accordingly, activity of a potential s of a myocardial surface mesh may be obtained using the measurement magnetic field B and the minimum variance spatial filter w (S155). Thus, the potential source may be extracted using the myocardial surface potential (S156). In addition, an ablation position of a route through which an abnormal circuit formed by the activity of the potential source passes may be extracted (S162).

On the other hand, there is a need to observe coherence of the activity of the potential source s at obtained different positions. In general, only a specific frequency component (f) is filtered to watch the coherence. The degree of the coherence p(f) is expressed by the Equation (7) below.

$$\hat{ρ} ( f ) = \frac{〈 \hat{s} ( {r}_{1} , f ) {\hat{s}}^{H} ( {r}_{2} , f ) 〉}{\sqrt{〈 {\vert \hat{s} ( {r}_{1} , f ) \vert}^{2} 〉 〈 {\vert \hat{s} ( {r}_{2} , f ) \vert}^{2} 〉}} \vert \qquad Equation ( 7 )$$

In the Equation (7), r1 and r2 represent positions of potential source activity, respectively; f represents a specific frequency; s(r,f) represents a complex Fourier component or complex Hilbert transform component of the potential source activity; and H represents a Hermitian transpose.

If the coherence is obtained with the Equation (7), a result of the coherence may be distorted due to interference of a peripheral potential source. Assuming that there is no coherence between interfering peripheral noise sources, coherence caused by a peripheral noise component always emerges as a real part. Accordingly, the interference of the peripheral noise component may be removed by observing only an imaginary part in the Equation (7) (S161).

In case of a typical minimum variance spatial filter, a current source presumes a current dipole. Therefore, power in triaxial direction is calculated at each point of a source space (heart position). Alternatively, power must be calculated with respect to a direction of previously assumed dipole. However, since a surface potential on the myocardium is a source in the present invention, it is sufficient to calculate only one scalar quantity. Moreover, in a conventional minimum variance spatial filter, power source must be estimated with respect to all mesh vertices by dividing the whole heart space into three-dimensional meshes. However, since a source power estimation point is limited to vertices of surface potential of a myocardial surface model in the present invention, the amount of calculation may be significantly reduced.

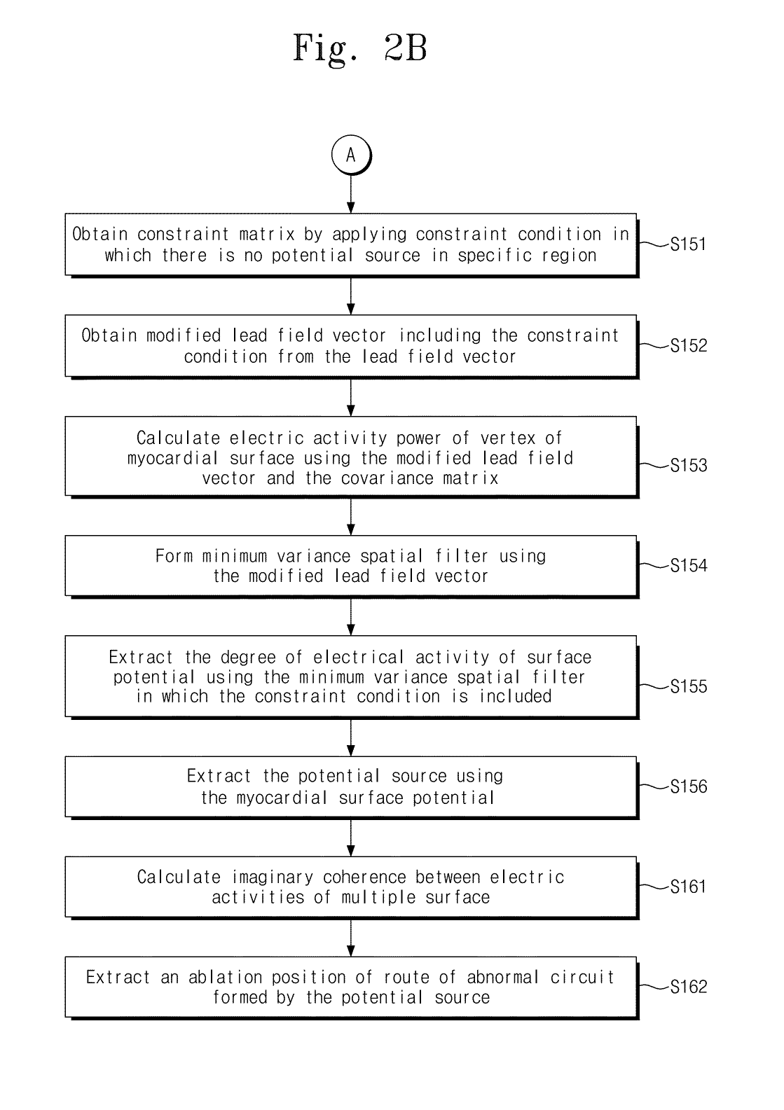

FIG. 3 illustrates a magnetocardiogram measuring device according to an embodiment of the present invention.

Referring to FIG. 3, a magnetocardiogram measuring device 14 is mounted inside a magnetic shielding room 10. The magnetocardiogram measuring device 14 may include a 64-channel superconducting quantum interference device (SQUID). The SQUID may be disposed on a plane to measure fine current and a magnetic field of human body. The SQUID may operate at an extremely low temperature of −250 degrees centigrade or less. Thus, the magnetocardiogram measuring device 14 may be mounted inside cooling means 13 to measure current and a magnetic field. The cooling means 13 may receive a coolant from a coolant tank 15. The cooling means 13 may be disposed inside a gantry 12. The gantry 12 may adjust a distance between a measurement-target person and the magnetocardiogram 14.

A driving circuit 11 drives the magnetocardiogram measuring device 14. An amplifier & filter 16 is disposed inside an RF shielding room 17. A power source unit 18 may supply power to the amplifier & filter 16 and the like. A magnetocardiogram signal is transmitted to a controller 19 to be processed and analyzed.

A measured signal of the magnetocardiogram measuring device 14 emerges as a magnetocardiogram signal having a similar pattern to an electrocardiogram signal. The magnetocardiogram signal has P, Q, R, S, and T peaks that sequentially emerge. The magnetocardiogram may include a P wave, a QRS wave, and a T wave. In case of a patient who has atrial fibrillation, the P wave may be converted into an f-wave.

The magnetocardiogram measuring device 14 may be a 64-channel plannar-type first-order gradiometer system. Magnetocardiogram data may be generated by separating a period in which an f-wave, which is a reentry wave of atrial fibrillation, emerges during 30-second measurement.

The f-wave of atrial fibrillation may be separated from ventricular excitation waves (QRS wave and T wave) using the independent component analysis or the like. In addition, a specific pattern on time series may be separated and analyzed depending on time.

Since a minimum variance spatial filter generally uses a covariance matrix of a multi-channel measured value, second-order statistical variances such as variance size are used to separate current sources. In some cases, the second-order variables are not enough to separate P wave, QRS wave, and T wave that are specific waves of magnetocardiogram (MCG). Accordingly, independent component analysis employing higher-order statistical variables may be used beforehand to time-serially separate measured waveforms.

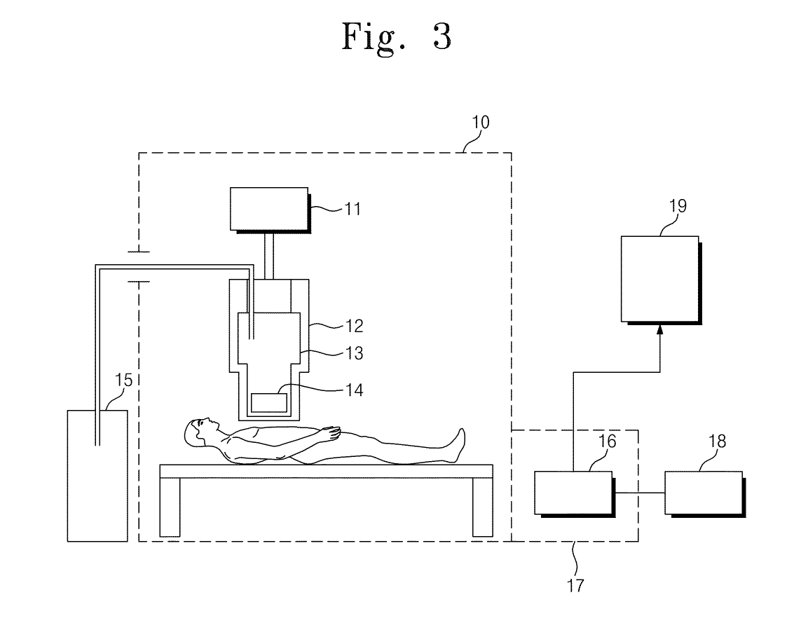

FIG. 4 illustrates counterclockwise periodic rotational excitation of myocardium according to an embodiment of the present invention.

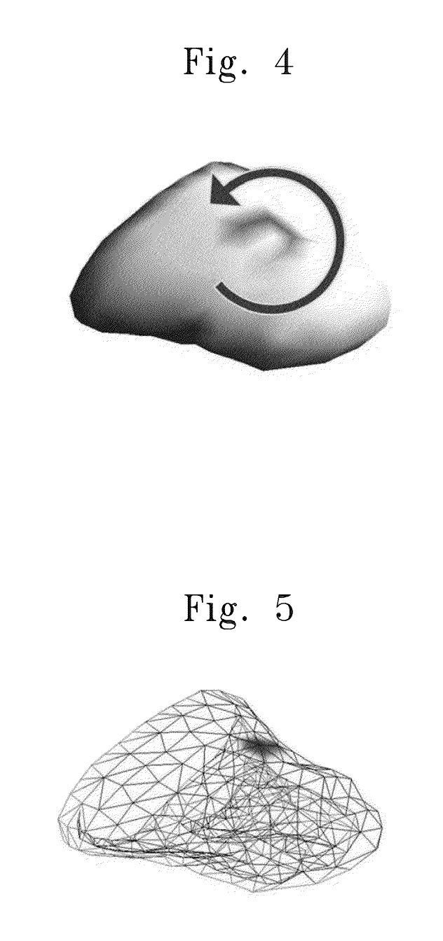

FIG. 5 illustrates a myocardial surface potential calculated through a method for mapping myocardial electric activity according to the present invention using a magnetic field or magnetocardiogram data calculated at respective positions where magnetocardiogram sensors are disposed. The magnetic field is generated from variation of a myocardial potential of FIG. 4.

Referring to FIGS. 4 and 5, a simple simulation test was performed to verify performance of a method for mapping myocardial electric activity according to the present invention. An electrocardiogram signal and a magnetocardiogram signal each include periodical P wave, QRS wave, and T wave. In case of a patient who has atrial fibrillation, the P wave may be converted into an f-wave.

A main cause of atrial arrhythmia generating an f-wave is a reentry wave. To simulate the reentry wave, a myocardial surface potential of a portion of a myocardial surface model was periodically excited while rotating counterclockwise. A magnetic field generated from variation of the myocardial surface potential was calculated at respective positions where magnetocardiogram sensors are placed. In addition, a random noise of 10 firms having a normal distribution was mixed with a calculated magnetic field corresponding to each sensor channel. The method for mapping myocardial electric activity according to the present invention was applied to the magnetic field data. As a result, strong source power (peak of the surface potential) emerges in the center position of the reentry wave.

A practically measured MCG-wave of a chronic cardiac arrhythmia patient was localized using a method for mapping myocardial electric activity according to an embodiment of the present invention. The measurement used a first-order gradiometer system, and the localization was performed by separating a period in which an f-wave emerged during 30-second measurement. During measurement of magnetocardiogram and CT, coordinate systems were made to match each other using patient's xiphoid and nipples as a landmark. Power of a potential source is extracted with respect to the f wave by means of the method for mapping myocardial electric activity according to the present invention.

A result of localization using a method for mapping of myocardial electric activity according to an embodiment of the present invention may be used in minimal ablation techniques for atrial arrhythmia. In a conventional surgery, after excising all possible parts where an abnormal circuit may be formed, they are stitched together. Meanwhile, in the above method, an ablation position of a route through which an abnormal circuit passes may be provided to increase a success rate of surgery and reduce the burden on patients and doctors.

A method for mapping of myocardial electric activity according to an embodiment of the present invention is a non-invasive technique because activity of a myocardial surface may be shown using magnetocardiogram. Thus, the method may also be used in prognosis observation after surgery of atrial arrhythmia.

According to the foregoing method for mapping of myocardial electric activity according to an embodiment of the present invention, an f wave formed by a reentry wave of the atrial arrhythmia is non-invasively measured in an isolation surgery of the atrial arrhythmia by using magnetocardiogram data or electrocardiogram data. This method can estimate a position by localizing the reentry wave causing the atrial arrhythmia. Thus, the method can help to perform an effective isolation surgery.

Although the present invention has been described in connection with the embodiment of the present invention illustrated in the accompanying drawings, it is not limited thereto. It will be apparent to those skilled in the art that various substitutions, modifications and changes may be made without departing from the scope and spirit of the present invention.

What is claimed is:

1. A method for mapping of myocardial electric activity, comprising:
measuring electrocardiogram data or magnetocardiogram data; and
mapping the degree of electric activity on the myocardial surface using the electrocardiogram data or the magnetocardiogram data;
wherein a signal source of the electrocardiogram data or the magnetocardiogram data is a myocardial surface potential that is scalar quantity; and
wherein the mapping uses a lead-field vector which represents sensitivity between the myocardial surface potential and the electrocardiogram or magnetocardiogram data, and a modified lead-field vector which combines a constraint matrix with a constraint condition where no potential sources exist in a specific region.

2. The method as set forth in claim 1, wherein mapping the degree of electric activity uses a minimum variance spatial filter, and
wherein the constraint condition suppresses the influence of correlated sources located at other regions to prevent interference generated from the correlated sources toward target sources to be estimated by the minimum variance spatial filter.

3. The method as set forth in claim 1, wherein mapping the degree of electric activity comprises:
forming a surface mesh to use a boundary element method as an electric conductor model of peripheral organs including the myocardium and thorax;
calculating a lead field vector between the myocardial surface potential and the electrocardiogram or magnetocardiogram data;
extracting a covariance matrix using multi-channel measured electrocardiogram or magnetocardiogram data;
obtaining a constraint matrix by applying a constraint condition in which there is no potential source in a specific region;
obtaining a modified lead field vector including the constraint condition from the lead field vector; and
calculating electric activity power of a vertex on the myocardial surface using the modified lead field vector and the covariance matrix.

4. The method as set forth in claim 3, wherein mapping the degree of electric activity further comprises at least one of:
forming a minimum variance spatial filter using the modified lead field vector;
extracting the degree of electric activity of a surface potential using a minimum variance spatial filter in which the constraint condition is included;
extracting a current source using the surface potential;
calculating imaginary coherence between electric activities among surface potentials; and
extracting an ablation position of a route of an abnormal circuit formed by the current source.

5. The method as set forth in claim 3, wherein calculating a lead field vector comprises:
calculating potentials on an organ surface generated by an unit potential on the myocardial mesh surface by a boundary element method; and
calculating a potential at an electrocardiogram electrode or a magnetic field at a magnetocardiogram sensor from the organ surface potential by a boundary element method.

6. The method as set forth in claim 1, further comprising at least one of:
measuring MRI or CT to include the heart and thorax;
forming an electric conductor model of patient's individualized heart and organ using MRI or CT data; and
separating a specific magnetocardiogram or electrocardiogram waveform to localize using second or higher-order statistics such as independent component analysis

7. The method as set forth in claim 1, wherein mapping the degree of electric activity comprises:
forming a surface mesh to use the boundary element method as the electric conductor model of a peripheral organ including the myocardium and thorax;
calculating a lead field vector between the myocardial surface potential and the electrocardiogram or magnetocardiogram data;
extracting a covariance matrix using the multi-channel measured electrocardiogram or magnetocardiogram data;
obtaining a constraint matrix by applying a constraint condition in which there is no potential source in a specific region;
obtaining a modified lead field vector including the constraint condition from the lead field vector;
forming a minimum variance spatial filter using the modified lead field vector; and
extracting the degree of electric activity of a surface potential using the minimum variance spatial filter in which the constraint condition is included.

8. The method as set forth in claim 7, wherein mapping the degree of electric activity comprises at least one of:
extracting a current source using the surface potential;
calculating imaginary coherence between electric activities of multiple surface potentials; and
extracting an ablation position of a route of an abnormal circuit formed by the current source.
